(19) United States
(12) Patent Application Publication
(10) Pub. No.: US 2021/0142878 A1
(43) Pub. Date: May 13, 2021

(54) Patient-Facing App for Health Literacy and Numeracy

(71) Applicant: Roberta D. Powell, Cranston, RI (US)

(72) Inventor: Roberta D. Powell, Cranston, RI (US)

(21) Appl. No.: 17/248,300

(22) Filed: Jan. 19, 2021

Related U.S. Application Data
(62) Division of application No. 16/394,020, filed on Apr. 25, 2019.

(51) Int. Cl. G16H 20/10; G16H 50/30; G16H 70/40

(52) U.S. Cl. CPC G16H 20/10; G16H 70/40; G16H 50/30

(57) ABSTRACT

A patient-facing digital platform designed to promote health literacy and numeracy that helps patients access, interpret, process, contextualize and act on personal health data so that they may manage their health conditions. The platform interprets and simplifies personal health data into simple illustrations; provides medication-interaction and substance-interaction information and medication-duplication information; offers health-education videos; and cross-references drugs, interactions, allergies and personal data to generate actionable information according to each person's profile. The app also translates medication information into various languages.

This application is a Divisional of application Ser. No. 16/394,020.

### TECHNICAL FIELD

The invention relates to systems and methods of interpreting health information and more particularly to interpreting, translating, cross-referencing, and generating actionable health data for laypersons. CPC schemes may include: Patient record management; Office automation, e.g. computer aided management of electronic mail or groupware; Social work; Time management, e.g. calendars, reminders, meetings or time accounting; ICT specially adapted for the handling or processing of patient-related medical or healthcare data for patient-specific data, e.g. for electronic patient records; and Computer-assisted prescription or delivery of medication, e.g. prescription filling or compliance checking.

### BACKGROUND

Most patients in the United States lack sufficient understanding of the health information surrounding their medical diagnoses and conditions. Health information includes prescription dosages and instructions; lab reports; patient literature; prescribed medications; over-the-counter medications; possible interactions, with certain foods, alcohol and street drugs; overdosage; and warnings and side effects.

According to Communicate Health (communicatehealth.com), “Only 10% of adults have the skills needed to use health information.” The remaining 90% lack the knowledge to understand and contextualize health information. The Healthcare Information and Management Systems Society states that “The ability to contextualize health information is a learned behavior; acquired through formal instruction, or in medical/nursing school. Poor health numeracy and literacy skills are exacerbated by the lack of patient health education, customarily provided by registered nurses. However, due to financial constraints and a general nursing shortage, often nurses have no time to provide health education to patients. As a result, the interpretation and contextualization of health data is predominately performed by the clinicians (MD, NP, PA), who spend less than ten minutes face-to-face with their patients, leaving little time for education and dialog.” As a result, patients find themselves unable to make informed decisions about dosage, or to adhere to a prescription regimen.

Americans of various educational levels face difficulty understanding written instructions or warning labels. For example, a patient may not know that the written prescription instructions “Take 3 times per day” actually means “Take every 8 hours.” Patients may also be unable to fully understand the implications of their diagnoses or health conditions, and may consequently fail to make appropriate lifestyle and behavioral decisions. The result is worsening conditions and, in the language of hospital administrators, poor patient outcomes.

Healthcare consumers depend on clinicians or pharmacists to identify medication or drug-class interactions or contraindications, or duplicate medication of differing names that may lead to over-dosage. When that fails, there is no single, accessible and reliable tool that interprets and/or clarifies medication instructions, interactions, and/or associates a prescription drug name with an over-the-counter medication name.

According to a recent study published in the journal Clinical Toxicology, “There is room for improvement in product packaging and labeling. Dosing instructions could be made clearer, especially for patients and caregivers with limited literacy or numeracy. One-third of medication errors resulted in hospital admission.” Studies have shown that patients with poor literacy have difficulty understanding medication labels.

The problem is more acute among low-literacy patients and patients for whom English is a second language. This sector struggles to interpret health data much more than those versed in healthcare or those fluent in English.

According to Univision, the Hispanic population alone accounts for over $23 billion in prescription drug sales in the United States annually, yet few, if any, pharmacy chains translate the medication labels or instructions to Spanish. The U.S. Federal government does not require pharmacies to translate prescription medication labels for non-English speakers. There is no easily accessible and reliable tool that translates, interprets and/or clarifies medication instructions and interactions for Limited-English-Speaking Patients (LEP) or those who do not speak English.

Non-prescription or “street” drugs and/or alcohol are sometimes taken simultaneously with prescription drugs. Most patients are unaware that any two of these drugs may interact, sometimes dangerously. Nor are patients aware that street drugs and alcohol may interact with each other, or be contraindicated with an existing health condition. Increased cannabis use in states where it has been legalized warrants assessment of contraindications and interactions with other drugs. The use of opioids presents an additional example of the use of non-prescription drugs.

Polypharmacy is the concurrent use of multiple medications by a patient. In a 2014 report the National Institutes of Health (NIH) stated that “polypharmacy, defined as the use of multiple drugs or more than are medically necessary, is a growing concern for older adults.” Older adults with cognitive decline are particularly vulnerable to incorrect medication self-administration. According to the NIH, “Specifically, the burden of taking multiple medications has been associated with greater health care costs and an increased risk of adverse drug events (ADEs), drug-interactions, medication non-adherence, reduced functional capacity and multiple geriatric syndromes.”

A prescription drug may differ in name depending on how it is sold. Pharmaceutical companies brand their drugs, with no nomenclature that links their brand name with the original name. For example one brand name for metformin is Glucophage. Once these drugs are sold as generics, a discount pharmacy chain might give them yet another name. Over-the-counter (OTC) medications are also sold under varying names. Because many consumers do not know the various names of a drug, they may inadvertently take multiple doses of it.

Health literacy is the ability to grasp and interpret health information and data to make informed health decisions. Health literacy includes the elements of aural literacy, mint literacy, numeracy and eHealth literacy. Aural literacy is the ability to understand what is heard. Print literacy is the ability to understand or write the written word. Numeracy is the ability to understand numerals, calculations, logic and interpretation of numerical content. E-Health literacy refers to the ability to navigate web-based and computer-based content.

Numeracy, in general, refers to the ability to use mathematical concepts and methods. Innumeracy, in general, refers to the inability to use mathematical concepts and methods.

Health numeracy is the capacity to access, understand, process and interpret data in order to manage one's health or to make health-related decisions.

The self-management of chronic disease requires adequate health-numeracy skills. Health innumeracy may result in a patient's inability to interpret and contextualize data about their health; a difficulty making informed decisions, which can lead to a worsening of symptoms or health conditions.

In the context of this disclosure, “medication” and “medicine” refer to prescription medications, vitamin supplements, over-the-counter (OTC) medications, brand-name drugs and generic drugs. “Substance” refers to non-prescription medications; alcohol; legal or illegal (“street”) drugs; or duplicate drugs of differing names.

A “machine-readable medium storing a program for execution by processor unit of a device” is commonly referred to as an application or app. Hundreds of apps offer health information and maintenance, but each app is specialized and limited by health condition. For example, blood-pressure monitoring, glucose-level monitoring, calorie counting or exercise regimentation apps are abundant in the field, but none provide qualitative or quantitative interpretation of health values or medications nor do they warn against potential interactions or duplicate drugs of differing names.

The “app” in this disclosure is called “Q2Q,” and is referred to herein as “the app.”

### SUMMARY

A system and method in the form of a patient-facing app, accessible via a smartphone, tablet and computer that helps people access, interpret, understand and contextualize personal health data so that they may manage their health conditions. The app interprets and simplifies personal health data, converting it into simple, color-coded illustrations; explains particular health information through animated videos; and provides evidence-based, relevant health education with behavioral suggestions. It checks for medication interactions and/or duplications between generic and name-brand drugs; between prescribed drugs and OTC drugs; between drug classes; between prescribed drugs and “street” drugs (also referred to here as “substances”) such as cannabis and opioids; and between drugs and alcohol and other substances. The app interprets medicine labels as they relate to a database of chronic conditions and to a user's entered conditions; suggests comparable medication alternatives to contraindicated medicines or substances; and provides warnings of potential overdose when both generic, name-brand or OTC drugs are input as prescribed or input as taken simultaneously. In instances of potential overdose, or of dangerous interactions between drugs or between drugs and foods, the app emits audio and text warnings indicating danger levels of mild, moderate or major.

The app performs immediate, one-to-many comparisons across contexts of drug interactions, health conditions and allergies. It analyzes user input and returns information about interactions, side effects and prescription dosages vs. OTC dosages, as well as information about chronic or acute health conditions.

The app offers relevant health education in the form of simple graphical representations; simple text explanations; and “explainer” videos.

The app integrates with electronic medical records (EMRs) through their APIs and via secure login.

The app's “dashboard” window includes health numbers such as past lab values as well as current medications that may be downloaded from the patient's electronic health record or entered by the patient.

The platform includes a program for receiving input in various ways, including:

Manual entry, via keypad, keyboard or similar text-entry means;

Scanned barcode entry, via a provided smartphone's camera;

Voice entry, via a provided smartphone's microphone;

Automatic download, via electronic medical record or patient portal.

Users first build a health profile by entering personal data including their age, weight, gender, allergies, and basic medical history. They may enter their medical history through their device keypad or load it from their electronic health record.

Once their profile is complete, a user may any of the above-mentioned interactions by inputting medications through any of the entry methods described above. The app cross-references their medications against the U.S. Federal Drug Administration (FDA)'s database and against the user's profile, checking allergies and medical history for possible interactions or contraindications. If an interaction is found, the app returns a color-coded alert message of minor, moderate or major concern.

When a user wants the app to determine interactions between prescribed, name-brand medications with prescribed, generic medications, they input that value through any of the entry methods described above. The app cross-references their medications against: 1. The U.S. Federal Drug Administration (FDA)'s database 2. The user's profile. Among these databases the app checks allergies and medical history for possible interactions or contraindications. If an interaction or duplication between generic and brand-name drug is found, the app returns a color-coded alert message of minor, moderate or major concern.

When a user wants the app to determine interactions of medications other than prescribed medications, such as street drugs, they input that value in text or voice format. The app cross-references their medications against: 1. the U.S. Federal Drug Administration (FDA)'s database; 2. A street-drug database such as the Center on Addiction's Commonly Used Illegal Drugs list; 3. The user's profile. Among these databases the app checks allergies and medical history for possible interactions or contraindications. If an interaction is found, the app returns a color-coded alert message of minor, moderate or major concern.

When a user wants the app to determine potential allergic reactions to medications, they input the medication in text, voice, or scanned-barcode format. The app cross-references the drug against 1. the U.S. Federal Drug Administration (FDA)'s database; 2. An allergy database; 3. The user's profile. Among these databases the app checks allergies, personal gene variants, and medical history for possible interactions or contraindications. If an interaction is found, the app returns a color-coded alert message of minor, moderate or major concern.

When a user wants the app to determine whether an OTC medication or vitamin supplement is safe to take during pregnancy or nursing, they input that substance's value by scanning its barcode. The app cross-references the drug against 1. the U.S. Federal Drug Administration (FDA)'s database; 2. the user's profile. Among these databases the app checks allergies and medical history for possible interactions or contraindications. If an interaction is found, the app returns a color-coded alert message of minor, moderate or major concern.

Any alerts or warnings remain active until a user removes the interacting or contraindicated substance from their list of active medications.

The app accepts and delivers information in multiple languages via text or voice input. It also translates information into various languages using available digital/machine translation and via artificial intelligence. In some embodiments, the language used for information entry is specified by the user; in other embodiments the language is recognized by the program in the app. One skilled in the art understands that information typed, scanned, spoken, or downloaded may be interpreted by a program to determine the language of input. Once the language of the input is determined, data is output in the same language or in a language of the user's choosing.

The app uses artificial intelligence (AI) to analyze entered data, such as patient history, to extract, interpret and produce actionable data. Entered data is captured in a database. It uses character and voice recognition to extract relevant values from photographs of patient lab reports and verbal inquiries; analyze extracted data; cross-reference the data and produce actionable information in user-friendly graphical elements.

One skilled in the art understands the ability of AI to recognize and interpret spoken words, scanned images or text, and to output the information to a machine-readable medium.

Through a text-messaging component, the app communicates alerts to a user's specified responsible parties such as family members or friends. An example of data communicated are warnings of high blood pressure or low blood-glucose levels or of a dangerous drug interaction or overdose.

Other objects and features will become apparent from the following detailed description considered in conjunction with the accompanying drawings. Drawings are intended to illustrate rather than define the limits of the invention.

### BRIEF DESCRIPTION OF THE DRAWINGS

To assist those of skill in the art in making and using the disclosed invention and associated methods, FIGS. 1-13 show the user interfaces of an example embodiment of the present disclosure, as shown on a provided smartphone.

FIG. 1 is a plan view of a user interface screen as shown on a provided smartphone.

FIG. 2 is a plan view of three related user interface screens.

FIG. 3 is a plan view of three related user interface screens.

FIGS. 4-13 show the user interfaces of a second example embodiment of the present disclosure, as shown displayed on a provided smartphone.

FIG. 4 is a plan view of a user interface screen of a second embodiment of the disclosure.

FIG. 5 is a plan view of three related user interface screens of the embodiment of FIG. 4.

FIG. 6 is a plan view showing results of user-entered information of FIG. 4.

FIG. 7 is a plan view of a user interface screen of the embodiment of FIG. 4 in which an example of a search result appears.

FIG. 8 is a plan view showing translation options (at the top of the screen) of a user-interface screen of the embodiment of FIG. 4.

FIG. 9 is a plan view of a text-magnify option (at the top of the screen) of the user-interface screen of the embodiment of FIG. 4.

FIG. 10 is a plan view of an interpretation feature of the user-interface screens of the embodiment of FIG. 4.

FIG. 11 is a plan view of an interaction checker of the user-interface screen of the embodiment of FIG. 4.

FIGS. 12-15 are flowchart views of user interaction with a second iteration of the embodiment.

FIG. 12 is a flowchart of user interaction with the embodiment.

FIG. 13 is a flowchart of user interaction with the embodiment.

FIG. 14 is a flowchart of user interaction with the embodiment.

FIG. 15 is a flowchart of user interaction with an iteration of the embodiment.

### DESCRIPTION

In an embodiment 100, FIG. 1 shows the app's initial screen 110 for choosing a primary language, in this case English 136.

FIG. 2 shows the start 112 of a program. A program-feature choice 138 has been selected. A specific health value 140 is selected from the health-value selector 114, giving the further option of selecting a mode selector for entering data 116. Options for entering data are manual entry 142; scanned entry 144; and spoken entry 146.

FIG. 3 shows the app's manual-entry option 118 and a specific manual-entry example 148, 150. A scan-entry option 120 is shown on another app screen. In this case, for example, the user has scanned their lab results 152. A voice-entry option 122 is shown in a third app screen. In this case the user has spoken an entry 154.

In a second iteration 200, FIG. 4 a user may select from various medication-entry methods 210 including manual medication entry 234, photograph entry 236 or barcode-scan entry 238.

FIG. 5 200 shows screens that are the result of each choice. In the manual medication entry screen 212 the user types a medication or drug using an on-screen keyboard. In the camera-entry screen 214 the user has taken a picture of a medication label. In the scanned-barcode entry screen 216 the user scans a medication via their (provided) smartphone's camera.

FIG. 6 200 shows a manual-entry result screen 218. In this example Diphenhydramine has been selected and the display shows both the drug name and the OTC name (Benadryl). This is an initial indication to the patient that the entered medications are the same drug.

FIG. 7 200 shows an example of a search result 220.

FIG. 8 200 shows the translation screen 222, where one may choose to translate indications and usage 240; dosage and administration 242; dosage forms and strengths 244; or warnings and precautions 246. In one embodiment, a patient taking a prescription for diphenhydramine who scanned a barcode on a container of Benadryl would receive a warning that the prescription medication and OTC medication are the same drug and that an overdose was possible if both were to be taken together.

FIG. 9 200 shows the text-magnify option 224 in which options 248, 250, 252 are shown magnified.

FIG. 10 200 shows the option to request entered information to be explained in simple terms 226. The information that was entered 256 may be simplified by tapping an explanation button 254. Once that button is tapped, the entered information is re-interpreted in simplified terms 228.

FIG. 11 200 shows an interaction checker 230 with example medications 260, 262 entered and an interaction 258 determined. In this example the patient is notified that the two medications entered are the same drug and that the dosage should be checked to avoid overdose.

In FIG. 12 200, a flowchart illustrates the progression of steps from a user's perspective. Upon opening the app 264 on their device, a user selects a language 266 and then builds their basic profile 268 by entering their height, weight, medicines, allergies and aspects of their medical history, either by downloading it from a patient portal 280 or by typing it on their device keypad. They choose Medication Assistant 270, which leads to an input screen for entering medications 271. This screen is further illustrated in FIG. 13.

The flowchart in FIG. 13 200 shows events once the user chooses “Medication Assistant” 241. The dashboard 243 loads, showing the user's current medications and other drugs. In some embodiments, the app checks for connection to a patient portal and if found, allows the patient to log in to retrieve electronic health record information such as recent visits. The user enters a medication or substance to be interpreted 245. Options for input include manual input 247, in which the user types 253 the name of the medication, drug or substance; camera-scan 249, in which the user employs the (provided) camera app on their smartphone to photograph or import 255 a photograph of a medication label; and barcode-scan 251, in which a user scans the barcode 257 on their over-the-counter medication using the app's barcode-scanning feature into their smartphone using the smartphone's provided voice app. Once a medication or substance is entered, the app seeks confirmation 259. If incorrect, the app re-routes 261 to the medication-entry step 245. If the entered medication or substance is confirmed by the user as correct, the app generates information about that medication or substance 263 including indications; prescription name and OTC name, values and types of dosage; administration; contraindications; precautions and warnings; and comparable medication alternatives. They may add 269 a medication the current medications. If an entered medication or substance has contraindications or possible interactions with their current medications, a pop-up box 265 appears indicating warning. If an interaction or contraindication is dangerous, the app will emit a warning sound and message. Users may adjust the text size 267 of the generated results by using a graphical slider.

FIG. 14 illustrates a third example iteration, 300. Upon opening the app on their device a user selects a language 312 and a text size 314, and then may choose to build their basic profile 316 by entering their height, weight, medicines, allergies and aspects of aspects of their medical history, either by downloading it from a patient portal 324 or by typing it on their device keypad. From there the user proceeds to a “Medication Assistant,” 320 which begins with an input screen that starts a process (FIG. 15) for obtaining medication information.

FIG. 15, 300 shows events after the user chooses “Medication Assistant” 360. The dashboard 364 loads, showing the user's current medications. In some embodiments, the app checks for connection to a patient portal and if found, allows the patient to log in to retrieve electronic health record information such as recent visits. The user enters a medication or substance to be interpreted 366. Options for input include manual input 368, in which the user types 374 the name of the medication; camera-scan 370, in which the user employs the (provided) camera app on their smartphone to photograph or import 376 a photograph of a medication label; and barcode-scan 372, in which a user scans the barcode 378 on their over-the-counter medication using the app's barcode-scanning feature. Once a medication is entered, the app seeks confirmation 380. If incorrect, the app re-routes 382 to the medication-entry step 366. If the entered medication/substance is confirmed by the user as correct 380, the app generates information about that medication/substance 384 including indications; values and types of dosage; administration; contraindications; duplicate medication; precautions and warnings; and comparable medication alternatives (for example, if there is an interaction with acetaminophen, the app suggests ibuprofen). They may add 390 this medication to list of current medications. If the entered medication/substance has contraindications or possible interactions with their current medications, a pop-up box 386 will appear with this information. Users may adjust the text size 388 of the generated results by using a graphical slider.

These embodiments are understood to be exemplary and not limiting.

1. A non-transitory computer-readable medium storing instructions that when executed by a computer cause the computer to perform operations of a method comprising:
providing a selection of medication assistance; and
displaying medication to be evaluated; and
displaying generic drug name and brand drug name to be evaluated; and
displaying substance to be evaluated; and
displaying manually entered data; and
displaying camera-scanned data entry; and
displaying barcode-scanned data entry; and
presenting a medication-information page relating to the entered data; and
generating an explanation of the indications and contraindications in text format in the operative language; wherein
a medication is described and a mode of data entry is chosen, and the medications entered by manual data entry are interpreted and compared for medical interactions, substance interactions, medication duplications; the results generate instructions as a graphic image in combination with explanation of the image in text format and video format, in the operative language.

2. The non-transitory computer-readable medium storing instructions of claim 1 that when executed by a computer cause the computer to perform operations of a method further comprising:
interpreting and generating information relating to substance interactions, in text format, in the operative language.

3. The non-transitory computer-readable medium storing instructions of claim 1 that when executed by a computer cause the computer to perform operations of a method further comprising:
interpreting and generating information relating to duplicate dosage of identical drugs of differing names and administration and generating warnings in text format in the operative language.

4. An apparatus and method for interpreting health information and generating instructions about substance interactions in a language of a user's choosing comprising:
a user interface for data entry of a patient's profile; and
a user interface for data entry into said patient's profile, of medications used by a patient; and
information derived from data entry is converted to non-transitory computer-readable medium storing instructions; and
said instructions compare said medications used by said patient to a database of medication interactions; and
said instructions define interactions between medications used by said patient; and
a numeral is assigned to an interaction according to the severity of said interaction; and
a graphic element is assigned to said numeral; wherein
the existence of and severity of an interaction between a medication and a medication in a patient's profile, is defined and depicted in a graphic image.

5. The apparatus and method of claim 4 further comprising:
a user interface for choosing an operative language; and
operations conducted in said operative language.

6. The apparatus and method of claim 4 further comprising:
a user interface for data entry of substances used by a patient; and
said instructions compare said medications used by said patient to a database of medication and substance interactions.
